(19) United States
(12) Patent Application Publication
(10) Pub. No.: US 2026/0130953 A1
(43) Pub. Date: May 14, 2026

(54) MARINE ANTIBACTERIALS AGAINST MRSA, VRE, ANTHRACIS BACILLUS AND OTHER GRAM-POSITIVE MICROORGANISMS

(71) Applicant: TREVOR PERCIVAL CASTOR, Arlington, MA (US)

(72) Inventor: TREVOR PERCIVAL CASTOR, Arlington, MA (US)

(21) Appl. No.: 18/942,707

(22) Filed: Nov. 10, 2024

(51) Int. Cl. A61K 35/74; A61K 47/02; A61K 47/10

(52) U.S. Cl. CPC A61K 35/74; A61K 47/02; A61K 47/10

(57) ABSTRACT

Multi-drug resistant (MDR) bacteria potentially pose a significant risk to people in the United States and all over the world; at the moment, the risk is the greatest where antibiotics are most commonly taken. The most notable MDR organisms are Methicillin-resistant Staphylococcus aureus (MRSA), Vancomycin-resistant Enterococcus faecium (VRE) and Carbapenem-resistant Pseudomonas aeruginosa. All three are associated with nosocomial infections. Embodiments of the present invention are directed to marine microorganism fractions and products for treating multi-drug resistant bacterial infections.

### GOVERNMENT SUPPORT

Embodiments of the present invention were conceived and reduced to practice without Federal sponsorship or funding.

### FIELD OF THE INVENTION

Embodiments of the present invention are directed to marine microorganism fractions and products for treating multi-drug resistant bacterial infections.

### REFERENCES TO OTHER PATENTS

This application discloses improvements and enhancements to Method of Fractionation of Biologically-Derived Materials Using Critical Fluids in U.S. Pat. Nos. 5,854,064 and 6,569,640 to Castor which are hereby incorporated by reference in its entirety.

This application discloses improvements and enhancements to Method and Apparatus for Isolating Therapeutic Compositions from Source Materials in U.S. Pat. No. 5,750,709 to Castor which are hereby incorporated by reference in its entirety.

This application discloses improvements and enhancements to Method and Apparatus for Extracting Taxol from Source Materials in U.S. Pat. No. 5,440,055 to Castor which are hereby incorporated by reference in its entirety.

### BACKGROUND OF THE INVENTION

Multi-drug resistant (MDR) bacteria potentially pose a significant risk to people in the United States and all over the world; at the moment, the risk is the greatest where antibiotics are most commonly taken. The most notable MDR organisms are Methicillin-resistant Staphylococcus aureus (MRSA), Vancomycin-resistant Enterococcus faecium (VRE) and Carbapenem-resistant Pseudomonas aeruginosa. All three are associated with nosocomial infections.

No population is more vulnerable to multi-drug resistance that those admitted to hospital wards. In the United States alone, more than 2.8 million individuals are infected and 35,000 die each year from hospital acquired multi-drug bacteria. MRSA, specifically community acquired MRSA (CA-MRSA), has emerged as a significant and growing health threat to the public at large. CA-MRSA has acquired increased virulence and pathogenicity and it increasingly causes aggressive infections in young, healthy people.

### SUMMARY OF THE INVENTION

Embodiments of the present invention are directed to marine microorganism fractions and products for treating multi-drug resistant bacterial infections.

One embodiment of the present invention is directed to the unique source material available. Taking advantage of the diverse marine environment, we have established a library of more than 1,500 unique marine microorganisms. The marine environment represents a virtually untapped resource for the discovery of new anti-MDR compounds.

Another embodiment of the present invention is directed to innovative extraction and purification methodology. A major limiting factor in the identification of industrial chemicals and therapeutic drugs from marine microorganisms is the discovery process itself. The conventional discovery process involves the fermentation of the marine microbes and the extraction of secondary metabolites from the fermentation broth.

In order to enhance the discovery process, we have developed and patented a supercritical fluid technology for first cellular disruption, and second, polarity-guided fractional extraction of biomass such as marine microorganisms. Some results of our work with this technology are described in detail in the referenced patents.

Our embodiments in this invention thus reside in the diversity of marine microorganisms being sourced for novel anti-MDR therapeutics, the enabling supercritical fluid technology platforms being used for drug discovery, isolation and manufacturing, and the innovative screening to identify compounds that may be synergistic with FDA approved antibiotics.

Embodiments of the present invention are directed to methods of using supercritical fluids for isolating and manufacturing antibacterial for use as a therapeutic to treat MRSA, VRE, Anthracis bacillus and other Gram-positive microorganisms.

Embodiments of the present invention are directed to marine microorganism fractions that are active against multi-drug resistant bacteria including Methicillin resistant Staphylococcus aureus (MRSA) and Vancomycin resistant Enterococcus faecium (VRE).

Embodiments of the present invention are directed to marine microorganisms that are active against multi-drug resistant bacteria including Methicillin resistant Staphylococcus aureus (MRSA) and Vancomycin resistant Enterococcus faecium (VRE).

Embodiments of the present invention are directed to products from marine microorganisms that are active against multi-drug resistant bacteria including Methicillin resistant Staphylococcus aureus (MRSA) and Vancomycin resistant Enterococcus faecium (VRE).

These and other features and advantages of the present invention will be apparent to those skilled in the art upon viewing the figures and reading the detailed description that follows.

### BRIEF DESCRIPTION OF THE FIGURES

FIG. 1 depicts in schematic form an apparatus embodying features of the present invention;

FIG. 2 depicts inhibition growth curves several marine microorganism fractions against MDR Enterococcus faecium ATCC 51559

FIG. 3 depicts the chemical structure of Magnesidin; and

FIG. 4 depicts the chemical structure of Prodigiosin.

### DETAILED DESCRIPTION OF THE INVENTION

Embodiments of the present invention will now be described in detail with respect to formulations and methods of treating multi-drug resistant bacterial infections as to the inventors' present best mode to practice the invention. This best mode may change over time as new considerations become known or available. Embodiments of the present invention are also subject to alterations and modifications such that the present teaching and description should not be considered limiting.

Multi-drug resistant (MDR) bacteria potentially pose a significant risk to people and patients in the United States and worldwide. The most notable MDR organisms are Methicillin resistant Staphylococcus aureus (MRSA), Vancomycin resistant Enterococcus faecium (VRE), and Pseudomonas aeruginosa. All three are associated with nosocomial infections. No population is more vulnerable to multi-drug resistance that those admitted to hospital wards. In the United States alone, 2.5 million individuals are infected and 35,000 die each year from hospital acquired multi-drug resistant bacteria. With fewer effective antibiotics available, infections caused by MDRs are becoming harder to treat and are associated with significantly higher rates of mortality and morbidity, longer hospital stays, and, consequently, greater healthcare costs. It is estimated that there were approximately 370,000 hospital stays for MRSA infections in 2005 in the U.S., and these stays cost an average of $14,000 versus $7,600 for all other stays. This equates to $2.4 billion in excess healthcare costs. This amount is likely to increase as MRSA screening programs continue to uncover higher infection rates.

MRSA, specifically community acquired MRSA (CA-MRSA), has emerged as a significant and growing health threat to the public at large. CA-MRSA has acquired increased virulence and pathogenicity and increasingly causes aggressive infections in young, healthy people. CA-MRSA, while only a few years ago relatively rare, it is now responsible for a majority of abscesses treated in emergency departments in the United States. One strain of CA-MRSA (MW2) was found to have 19 genes encoding virulence factors not found to date in other S. aureus genomes. By contrast, one virulence factor now found in virtually all strains of CA-MRSA is Panton-Valentine Leukocidin cytotoxin. This cytotoxin increases transmissibility of the pathogen by enabling it to breach mechanical barriers (skin, airway epithelium), and so result in extensive outbreaks among groups such as the homeless, students, inmates, and soldiers. The spectrum of diseases caused by CA-MRSA has broadened to include severe and necrotizing pneumonia more commonly, toxic shock syndrome, sepsis, necrotizing fasciitis, endocarditis, septic joints and osteomyelitis.

At the same time that problems of drug-resistant, pathogenic bacteria are spilling from hospital wards into every-day life and there is a growing need for effective new antibiotics to keep at bay increasingly aggressive bacterial pathogens, the pipeline for novel antibiotics is becoming exhausted. Since the 1970's, two novel classes of antibiotics have been approved for use in humans by the FDA: oxazolidinones, and cyclic lipopeptides, and resistance to these in strains of MRSA and VRE have already been encountered. With the emergence of fully glycopeptide resistant strains of MRSA in the U.S., there is concern that a devastating large-scale epidemic is increasingly possible, with high levels of mortality as occurred in the pre-antibiotic era when death rates from S. aureus bacteremia as high as 82% were documented.

The reasons for antibiotic drug resistance are thought to be many, and include antibiotic overuse, improper use such as prescribing antibiotics for a viral infection or premature termination due to lack of compliance, transfer of resistance from one bacterial strain to another and possibly, the use of antibiotic soaps and lotions that increase the exposure of bacteria to these antibiotics in the sewage system. The primary factors responsible for acquisitions of these infections are selective pressures due to antibiotic use, compromised resistance to disease among the hospital patients, and transmission of organisms from patient to patient by nurses, physicians, and other health care workers. The net result is an increased incidence in the proportion of drug-resistant bacteria in the hospital setting.

Prior to the discovery, development and use of antimicrobial agents, morbidity and mortality from bacterial infection were significant. In the early 1900's, the three leading causes of death were pneumonia, tuberculosis, and enteritis. These three infections were responsible for 40% of all deaths. The introduction and commercial use of antimicrobial agents has significantly advanced the fight against bacterial infection. However, the emergence of antimicrobial resistant bacteria is reversing their effectiveness, proving that bacteria will continue to evolve mechanisms to ensure their survival. As early as half a century ago, penicillin resistant strains of Staphylococcus aureus were identified. Since that time, the problem of antimicrobial resistance has snowballed into a serious public health concern, which is global in scope and crosses all economic and social boundaries.

aphylococci are primarily associated with the skin and mucous membranes of humans and animals, and most species are presumed to be harmless. Staphylococcus aureus is considered the most pathogenic species and causes a wide range of diseases, including endocarditis, osteomylitis, pneumonia, toxic-shock syndrome, scalded skin syndrome and food poisoning. This organism is of particular concern because it can easily establish resistance to a wide range of antibiotics. In the early 1950's the widespread distribution of β-lactamase resistance plasmids in S. aureus decreased the effectiveness of penicillin against this organism. In 1959, methicillin, a synthetic penicillin was introduced onto the marketplace. However, methicillin-resistant S. aureus (MRSA) was identified just one year later. MRSA is frequently resistant to other clinically relevant antibiotics including macrolides and aminoglycosides. Vancomycin, a glycopeptide was the drug of choice for treatment of methicillin resistant strains. However, organisms with glycopeptide intermediate resistance have now been identified. There are also S. aureus strains that have high levels of vancomycin resistance.

Enterococci are normal human microbiota found within the intestinal tract. Although they are not considered to be highly virulent, their ability to develop intrinsic resistance to broad-spectrum antibiotics allows them to form super infections in humans especially in the nosocomial setting. Vancomycin-resistant enterococci (VRE) were reported in Europe in 1988, and since then the proportion has increased to about 26% of isolates by 1999. The increase in resistance is from nosocomial sources and results in the establishment of a resistant microbial population. Linezolid, a new oxazolidinone antibiotic was approved by the FDA in April 2000. In clinical trials, VRE resistance to this antibiotic occurred rarely and was thought to be a solution to the treatment of VRE infections. However, infections due to vancomycin resistant Enterococcus faecium and methicillin-resistant Staphylococcus aureus, i.e., linezolid-resistant strains have now been identified in the clinical setting. This emphasizes the need for the discovery and development of new antimicrobial agents against VRE and other MDR bacteria.

The major antibiotics in current clinical use were discovered by screening terrestrial microorganisms, primarily actinomycetes that appear to have a Darwinian propensity to produce antimicrobials against human pathogens. Over the past decades, however, the discovery rate of new antibiotics from terrestrial sources has decreased. The dereplication rate for the terrestrial environment is estimated to be greater than 90%. In contrast, the marine environment represents a relatively unexplored resource for the discovery of new antimicrobial compounds. The extreme diversity of the marine environment with respect to salinity, temperature, pressure and nutritional availability, provides an environment that selects for a high level of genetic and molecular diversity among the microorganisms that exist there. Marine microorganisms have developed unique metabolic and physiological properties that allow them to survive in these extreme environments. Marine bacteria have the ability to incorporate halogens (Br⁻, Cl⁻, F⁻, and I⁻) found in high concentrations in seawater into secondary metabolites. This is important because many antimicrobial compounds are halogenated. One of the first metabolites isolated from a marine microorganism was a highly brominated pyrole antibiotic that contains 70% bromine by weight. Urauchamycin is an antimicrobial produced by marine Streptomyces, while andrimid and moiramide are antimicrobials isolated from marine Pseudomonas fluorescens.

One reason that antibiotic discovery has diminished relates to the falloff in lead molecules from traditional terrestrial sources, and the failure of synthetic, high throughput approaches to fill the gap. Since all but three classes of existing antibiotics are based upon natural products, it makes sense to look to sources of new biological diversity for novel and effective antimicrobials. Eighty percent of the world's plant and animal species are found in aqueous habitats. Among 34 fundamental phyla of life, 32 can be found in the oceans, 2 are found only on land, while 17 are found only in the ocean. Marine organisms are often more permeated by their environment which contains up to one million (10⁶) microorganisms and ten million (10⁷) viruses in every milliliter. The dry weight of a sponge is about 50% microorganisms; this is one of the reasons that sponges and related marine animals are among the best sources of novel marine natural products. They have evolved at the epicenter of a complex array of microbial interactions and co-evolution. It is thought that many of the bioactive compounds produced by marine animals such as sponges are manufactured by their microbial symbionts. Marine microbes represent about 10% of living matter on the planet and it is estimated that only 5% have been cultured using standard techniques. This extraordinary diversity of life and the degree to which it is unknown became evident with the publication in Science of Venter et al, Environmental Genome Shotgun Sequencing of the Sargasso Sea in 2004, which found 800 new genes coding for rhodopsins, almost triple the number previously known. Looking to the great reservoir of unexplored biodiversity locked up in the world's oceans for novel antibiotics right now makes sense for the two reasons alluded to above: increasing need and decreasing supply.

Up to now within marine habitats a great deal of attention has been paid to extremophilic organisms, those that flourish in ecological niches that were in many cases thought to be inhospitable and even incompatible with life. Perhaps because they are often protected by the hostility of their environments to other life-forms, and because they have had to evolve novel biology to survive under harsh environmental conditions, extremophiles have so far been more useful for their enzymes and other unusual proteins than for antibiotics and cell-growth modifying agents. This illustrates the idea that microbial community structure and the specific environments in which they grow may be expected to have a strong effect on the types of secondary metabolites produced. More generally, there is a growing recognition that future drug discovery in the ocean may be more effectively accomplished though intelligent bioprospecting. That is, through targeting biological systems in a selective fashion.

The discovery that marine organisms are a rich source of antimicrobial compounds is not entirely surprising because many algal and invertebrate phyla reside exclusively in the ocean. This discovery seems logical given that there are many resident soft-bodied organisms, lacking obvious structural defense mechanisms, produce secondary metabolites as a means of chemical defense. There is now evidence that many of these compounds are actually produced by resident symbionts. For example, the cytotoxic macrolide swinholide A and cyclic peptides are produced by symbiotic cyanobacteria. Marine microorganisms are thus being increasingly looked to as a source of novel bioactive compounds.

One embodiment of the present invention is directed to the unique source material available. Taking advantage of the diverse marine environment, we have established a library of more than 1,500 unique marine microorganisms. The marine environment represents a virtually untapped resource for the discovery of new anti-MDR compounds. Thanks to its extreme diversity with respect to salinity, temperature, pressure, and nutritional availability, the variety of animals, plants, and microorganisms that the oceans harbor is quite significant. Only a few thousand of these microorganisms have been isolated, and it is acknowledged that perhaps 5% of the resident microbial population is actually cultivable by present day technology. Marine microorganisms are thus being increasingly looked to as a source of novel bioactive compounds. Microorganisms in our collection have also been isolated from deep sea and near shore locations, halophilic ponds and mangrove swamps, as well as from sponges, corals, and other invertebrates that are known to produce bioactive compounds. Based on the data on morphological and colony characteristics and RAPD-PCR based DNA fingerprinting, the redundancy rate of our collection is less than 10%.

The chemical structures of bioactive compounds isolated from marine flora and fauna are often different from those found in terrestrial sources. The reason is that marine organisms possess a number of unusual biochemical features. This results from the unique natural environment of their habitat and, consequently, an ingenious variety of metabolic pathways adopted by these organisms. To support this claim, a number of novel compounds with profound anti-infectious activity have been isolated from marine biomass. Collectively taken, the conditions described above provide for a major pharmaceutical resource, which is currently virtually unscreened for its anti-MDR activity.

Another embodiment of the present invention is directed to innovative extraction and purification methodology. A major limiting factor in the identification of industrial chemicals and therapeutic drugs from marine microorganisms is the discovery process itself. The conventional discovery process involves the fermentation of the marine microbes and the extraction of secondary metabolites from the fermentation broth. For the extraction, the fermentation broth is vigorously contacted with an organic solvent such as butanol or ethyl acetate, so that compounds of potential interest can be transferred from the aqueous broth and cellular substrate to the solvent phase. This process usually results in the extraction of a complex mixture of analytes, which are then screened for specific activities such as cytotoxic, antibacterial or antiviral in the pharmaceutical and biotechnology industries.

The screening of a complex mixture of analytes is not always successful in sensitive screens for specific activities. Often, interfering compounds that mask specific activities or ubiquitous compounds that interact with all screens give false positives. This problem can be resolved by purification of the analytes. Such an approach is not only cost-prohibitive but will also result in biasing the isolation of analytes that occur in higher concentrations. Typically, bioactive compounds are found in trace quantities, e.g., the potent anti-cancer compound Bryostatin-1 is found at a level of 1 to 5 ppm in the bryozoan Bugula neritina. An excellent compromise will be to isolate partially purified fractions as long as the process is rapid, exhaustive and cost-effective.

Conventional organic solvents are not always ideal for biomass extractions. These solvents can be difficult to remove from the compounds potentially exhibiting bioactivity and thus can mask bioactivity in an assay. The solvation properties of conventional solvents cannot be readily modified and are thus cumbersome to use for the selective extraction of compounds of varying polarity. In many cases, the bioactive materials of interest may be sequestered within the substrate or other cellular structures and not accessible to extraction. Most organic solvents cannot penetrate these membranes without physical disruption by techniques such as grinding, sonication, and homogenization. Such steps are not traditionally utilized because of the additional time and incremental costs in sample preparation.

In order to enhance the discovery process, we have developed and patented a supercritical fluid technology for first cellular disruption, and second, polarity-guided fractional extraction of biomass such as marine microorganisms. Some results of our work with this technology are described in detail in the following patents “Method of Fractionation of Biologically-Derived Materials Using Critical Fluids,” U.S. Pat. Nos. 5,854,064 and 6,569,640.

In addition to screening partially purified marine molecules against MRSA and VRE, we screened these fractions in the presence of clinically relevant antibacterials e.g., ciprofloxacin, gentamicin or vancomycin, to evaluate potential synergism between approved drugs and novel marine molecules. The possibility exists that one compound will eliminate drug efflux mechanisms and the second drug will kill the bacterium, or the combination may work better at lower concentrations than the two separately, thus making cytotoxicity less a concern.

Our embodiments in this invention thus reside in the diversity of marine microorganisms being sourced for novel anti-MDR therapeutics, the enabling supercritical fluid technology platforms being used for drug discovery, isolation and manufacturing, and the innovative screening to identify compounds that may be synergistic with FDA approved antibiotics.

One preferred antibacterial composition is formed from a dried marine microorganism biomass. This dried biomass is placed in a vessel with carbon dioxide under supercritical, near-critical or critical conditions to form a saturated biomass powder. The carbon dioxide is separated from the biomass to form a carbon dioxide fluid extract containing an antibacterial composition.

Preferably, carbon dioxide is held at a temperature of 20-50 degrees Celsius (° C.), at a pressure of 1,000 to 4,000 psig. Preferably, the carbon dioxide has a modifier or cosolvent, in the sense that the cosolvent is carried in the carbon dioxide in the nature of a dissolved constituent. A preferred cosolvent is an alcohol, such as methanol or ethanol.

Aspects of the present invention employ materials known as supercritical, critical or near-critical fluids. A material becomes a critical fluid at conditions which equal its critical temperature and critical pressure. A material becomes a supercritical fluid at conditions which exceed both its critical temperature and critical pressure. The parameters of critical temperature and critical pressure are intrinsic thermodynamic properties of all sufficiently stable pure compounds and mixtures. Carbon dioxide, for example, becomes a supercritical fluid at conditions which equal or exceed its critical temperature of 31.1° C. and its critical pressure of 72.9 atm (1,070 psig). In the supercritical fluid region, normally gaseous substances such as carbon dioxide become dense phase fluids which have been observed to exhibit greatly enhanced solvating power. At a pressure of 3,000 psig (204 atm) and a temperature of 40° C., carbon dioxide has a density of approximately 0.845 g/cc and behaves much like a nonpolar organic solvent, having a dipole moment of zero Debyes.

A supercritical fluid displays a wide spectrum of solvation power as its density is strongly dependent upon temperature and pressure. Temperature changes of tens of degrees or pressure changes by tens of atmospheres can change a compound solubility in a supercritical fluid by an order of magnitude or more. This feature allows for the fine-tuning of solvation power and the fractionation of mixed solutes. The selectivity of nonpolar supercritical fluid solvents can also be enhanced by addition of compounds known as modifiers (also referred to as entrainers or cosolvents). These cosolvents are typically somewhat polar organic solvents such as acetone, ethanol, methanol, methylene chloride or ethyl acetate. Varying the proportion of cosolvent allows wide latitude in the variation of solvent power.

Supercritical, near critical and critical fluids can exhibit liquid-like density yet still retain gas-like properties of high diffusivity and low viscosity. The latter increases mass transfer rates, significantly reducing processing times. Additionally, the ultra-low surface tension of supercritical fluids allows facile penetration into microporous materials, increasing extraction efficiency and overall yields.

A material at conditions that border its supercritical state will have properties that are similar to those of the substance in the supercritical state. These so-called “near-critical” fluids are also useful for the practice of this invention. For the purposes of this invention, a near-critical fluid is defined as a fluid which is (a) at a temperature between its critical temperature (T_{c}) and 75% of its critical temperature and at a pressure at least 75% of its critical pressure, or (b) at a pressure between its critical pressure (P_{c}) and 75% of its critical pressure and at a temperature at least 75% of its critical temperature. In this definition, pressure and temperature are defined on absolute scales, e.g., Kelvin and psia. To simplify the terminology, materials which are utilized under conditions that are supercritical, near-critical or exactly at their critical point with or without polar cosolvent will jointly be referred to as “SuperFluids or referred to as “SFS.”

SuperFluids can be used for the fractional extraction and manufacturing of highly purified antibacterial compounds.

Embodiments of the present invention are directed to methods of using supercritical fluids for isolating and manufacturing antibacterial for use as a therapeutic to treat MRSA, VRE, Anthracis bacillus and other Gram-positive microorganisms.

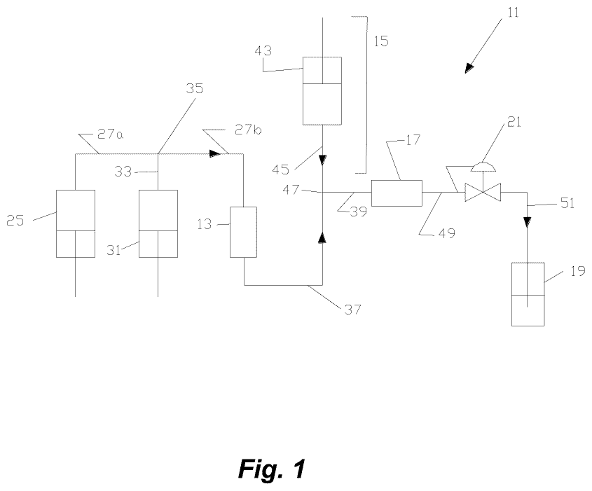

The present method and apparatus will be described with respect to FIG. 1 which depicts in schematic form the fractionation apparatus, generally designated by the numeral 11.

Polarity-guided SuperFluids fractionation can be carried out on the dried and fresh marine microorganisms. SuperFluids CXF fractionations can be carried out on an automated extractor or a manual version of the same. As shown in FIG. 1, this is a dual pump system, utilizing syringe pump 25 for neat critical fluid (e.g., CO₂) and syringe pump 31 for cosolvent (e.g., ethanol).

After loading marine microorganisms into a cartridge on the cartridge holder 17, the fractionation procedure can start. For example, the system will be brought to 3,000 psig and 40° C., and extracted for 10 minutes with pure CO2. This fraction will be collected in ethanol in a glass vial, numbered 19 in FIG. 1. The extraction parameters will be then set to: Supercritical CO2 at 3,000 psig and extraction temperature 40° C., step extractions with ethanol as cosolvent at 5, 10, 20, 30 and 40 vol % each step being 10 min. Each biomass sample will yield 6 fractions, which will be collected in ethanol in separate glass vials. The fractions will be dried under vacuum in a SpeedVac, and analyzed by HPLC for gingerols, zingerone, and shogaol content. Conditions which provide the highest antibacterial activities are favored for manufacturing larger quantities.

### EXAMPLES

### Example 1: Marine Microorganism Library

We have established a library of more than 1,500 unique marine microorganisms from diverse environments, including deep-sea sediments to shallow water mangrove swamps, tropical waters to temperate oceans, hydrothermal vents as well as normal saline to hypersaline conditions. These marine microorganisms include marine invertebrates-bryozoans, sponges, corals and tunicates; and marine vertebrates-shark and fish.

Marine microorganisms were isolated from U.S. territorial waters and consists of 70% Gram-negatives; 10% Gram-positives excluding actinomycetes; 10% actinomycetes; and 10% yeast and fungi. 10% of the collection consists of obligate halophilic bacteria. Microorganisms were also isolated from sponges, corals, and other invertebrates that are known to produce bioactive compounds. There is now evidence that many of these compounds are actually produced by resident symbionts. Experiments conclude that the cytotoxic macrolide swinholide A and cyclic peptides are produced by symbiotic cyanobacteria.

Marine microorganisms are characterized by one or more of the following techniques: (i) conventional microbiology including cell morphology, colony morphology, color, Gram stain, motility and substrate utilization; (ii) fatty acid methyl ester (FAME) profiles including an expanded MIDI database for a subset of the marine microorganisms library; (iii) DNA fingerprinting by the RAPD (random amplification of polymorphic DNA) technique utilizing polymerase chain reaction (PCR) for identification and redundancy minimization; and (iv) for certain hit marine microorganisms, molecular taxonomy via DNA sequencing of the 16 ssuRNA gene for similarity rank and blast comparisons with ribosomal sequence databases. Based on the data on morphological and colony characteristics as well as RAPD-PCR based DNA fingerprinting, the redundancy rate of our collection is <10%.

We also developed and utilized proprietary fermentation techniques that mimic the natural saline marine environment in order to enhance isolation of bioactive compounds and allow large-scale manufacture of novel anti-infectives and anticancer drugs. The microorganisms (bacteria, Actinomyces, yeasts and fungi) were fermented in at least four different media designed to maximize the diversity of secondary metabolites being generated.

### Example 2: Marine Microorganisms Fermentation

Marine microorganism cultures are stored in glycerol stocks at −80° C. Isolate descriptions typically include colony characteristics, motility, Gram stain, cell morphology and DNA fingerprinting. Selected marine microorganisms are withdrawn from storage and streaked on saltwater agar medium (ASA) to verify purity. Their identities are verified against our in-house database. The marine isolates are grown under optimized fermentation conditions in 10-liter quantities so that substantial amounts of biomass are available for isolation purposes. The media to be used for marine fermentation is prepared in an artificial seawater (ASW) base. The advantage of using ASW is that its chemical composition will remain consistent, as compared with natural seawater. GP2 formulation ASW is used for all fermentations. GP2 is chosen because it is closest in chemical composition to natural seawater and, since it is prepared as two solutions that are mixed after autoclaving, it does not precipitate. This allows use of full-strength ASW for marine fermentations. GP2 seawater has been successfully utilized in our marine microorganism program.

Marine Microorganism Fermentation Media: APP214 and other marine microorganisms were maintained as frozen stock cultures prepared in 10% glycerol and stored at −80° C. Prior to initiating liquid cultures, the stock culture was streaked on saltwater agar and examined by colony characteristics, Gram-staining, and cell morphology, to ensure purity.

The media used for fermentation was prepared in an artificial seawater (ASW) base. The advantage of using ASW is that its chemical composition remains consistent, as compared with natural seawater. GP2 formulation ASW was used for all fermentations. GP2 was chosen because it has the closest chemical composition to natural seawater and, since it is prepared as two solutions that are mixed after autoclaving, it does not precipitate. This allowed the use of full-strength ASW for marine fermentations. GP2 seawater has been successfully utilized in our marine microorganism program. The composition of the GP2 artificial sea water is listed in Table 1.

TABLE 2
Composition of GP2 Artificial Sea Water (per Liter)

| Chemical | Amount (grams) | Chemical | Amount (grams) |
|---|---|---|---|
| NaCl | 23.9 | Na₂SO₄ | 4 |
| KCl | 0.698 | NaHCO₃ | 0.193 |
| KBr | 0.1 | Na₂B₄O₇•10H₂O | 0.039 |
| MgCl₂•6H₂O | 0.108 | CaCl₂•2H₂O | 1.5 |
| SrCl₂•6H₂O | 0.0243 | NaH₂PO₄•H₂O | 0.0128 |
| Ferric citrate•H₂O | 2.42 × 10⁻⁵ | Na₂MoO₄•2H₂O | 8.3 × 10⁻⁵ |
| KI | 2.18 × 10⁻⁵ | ZnSO₄•7H₂O | 2.18 × 10⁻⁵ |
| NaVO₃ | 6.1 × 10⁻⁶ | MnSO₄•H₂O | 6.08 × 10⁻⁷ |
| Urea | 4.47 × 10⁻² | Thiamine•HCl | 1.95 × 10⁻³ |
| Biotin | 9.99 × 10⁻⁷ | Cyanocobalamine | 9.77 × 10⁻⁷ |

Cultures for routine use were maintained on the appropriate agar slants or plates kept at 4° C. Cultures were routinely examined by colony characteristics, Gram-staining, and cell morphology, to ensure axenicity. Four liquid fermentation media differing in chemical composition were used to grow any given culture. Use of more than one medium helps to maximize the diversity of secondary metabolites. The media described below are high or low in particular nutrients, specifically carbon and nitrogen. Carbon sources were glucose, glycerol, or Na acetate. Nitrogen sources were peptone, yeast extract, and beef extract. The following protocol for the four medium compositions was used: (a) HCLN: 0.5% glucose, 0.5% glycerol, 0.2% peptone, and 0.2% yeast extract [Media R]; (b) LCHN: 0.2% glucose, 0.2% glycerol, 0.1% Na acetate, 0.8% peptone, 0.2% yeast extract [Media S]; (c) LCLN: 0.2% glucose, 0.2% peptone, 0.2% beef extract [Media T]; and (d) HCHN: 0.5% glucose, 0.5% glycerol, 0.2% Na acetate, 0.8% peptone, and 0.2% beef extract [Media U]. Marine fermentation media were prepared in GP2 artificial sea water per the listing in Table 1 above.

Initial Growth of APP214 and Other Marine Microorganisms: Isolated colonies were used to initiate 5 ml starter cultures of 5 ml of each of four marine media R, S, T, and U prepared in 10 ml flasks. Each flask was sterilized by autoclaving at 121° C. and 1.1 kPa for 15 min. Each flask was incubated for 4 days at 37° C. on a shaker at 250 rpm (2.5 cm stroke), to provide adequate oxygen levels during the growth period. These starter cultures were used to inoculate the four 250 ml volumes of each media type in one-liter flasks. The flasks were incubated at 25° C. on a shaker 250 rpm (2.5 cm stroke) for 7 days. The 7-day incubation period ensured that the culture was in stationary phase of growth, where most of the secondary metabolite production is expected to take place. At the end of the seven-day incubation period, the cultures grown in the same media type were combined into two 500 ml batches. Fermentations were carried out in one-liter Erlenmeyer flasks to provide adequate biomass for isolation of the active compound. Each flask contained 500 ml of the appropriate medium, and was sterilized by autoclaving at 121° C. and 1.1 kPa for 15 min. One batch was utilized for antimicrobial screening. The second 500 ml fraction of each culture was split and extracted.

A starter culture of each organism is prepared by inoculating the marine microorganism from isolated colonies into 50 ml of marine medium R in a 250 ml Erlenmeyer flask. The flask is shaken at 25° C. and 250 rpm (2.5 cm stroke), until growth is observed. The incubation temperature of 25° C. is sufficient to support growth of the majority of the microorganisms in our culture collection. These cultures are examined by Gram-staining to ensure purity. This starter culture is used to inoculate a 10-liter fermenter filled with the same media type. The initial parameters are 25° C., with 1,000 cc/minute air, 100 rpm of agitation and an incubation time of 7 days. The 7-day incubation period ensures that all of the cultures reach the stationary phase of growth, when most of the secondary metabolite production is expected to take place. The culture is monitored microscopically on a periodic basis by Gram-staining to confirm their axenic nature.

Large-Scale Fermentation of Marine Microorganism APP-214 and Other Marine Microorganisms. The marine isolate APP-214 and others were grown under optimized fermentation conditions in 10-liter quantities to provide adequate biomass for isolation of the active compound. APP-214 et al. were streaked on saltwater agar from a frozen stock culture prepared in 10% glycerol and stored at −80° C. Prior to initiating liquid cultures, the stock cultures were examined by colony characteristics, Gram-staining, and cell morphology, to ensure purity. Marine medium R was used for growth of APP-214 because this gave the highest bioactivity of each of the four respective media used in the initial screening. Isolated colonies were used to initiate a starter culture of 50 ml of marine medium R prepared in a 250 ml flask. The flask was incubated overnight at room temperature on a shaker at 250 rpm (2.5 cm stroke), to provide adequate oxygen levels during the growth period. This starter culture was used to inoculate the ten-liter fermenter. The fermenter was incubated at 30° C. with 100-150 rpm agitation and 1,500 cc/min air input for three days.

Although this temperature is above that found in the marine environment, the incubation temperature of 30° C. provided optimal growth of this organism. Growth curves of this organism demonstrate that this culture reaches stationary phase within 24 hours, and there is no difference in the activity in the cell pellet after 24 hours. A 3-day incubation period ensured that the culture was in stationary phase of growth, where most of the secondary metabolite production is expected to take place. At the end of the 3-day incubation period, the microbial cell mass was harvested by centrifugation at 10,000×G for 15 minutes, lyophilized and divided into two aliquots, one for organic extraction and the other for an aqueous fraction and SuperFluids fractionation of the cell pellet. The aliquots were stored at −80° C. until further use.

### Example 3: Marine Microorganisms Fractionation

In preliminary studies, 4,800 extracts from 400 marine microorganisms were utilized as a starting point for a systematic search for the discovery of antimicrobial compounds for oral care (plaque, caries and gingivitis).

Marine microbes were first fermented by methods that mimic the natural saline marine environment in order to enhance isolation of bioactive compounds. The microorganisms were fermented in four different saline media designed to maximize the diversity of secondary metabolites and enzymes. The media differed in the concentrations and type of added carbon and nitrogen sources. Carbon sources were glucose, glycerol, or Na acetate. Nitrogen sources were peptone, yeast extract, or beef extract. Marine fermentation media were prepared in GP2 artificial sea water. Fermentations were carried out in 500 ml Erlenmeyer flasks. Each flask contained 100-125 ml of the appropriate medium, and was sterilized by autoclaving at 121° C. and 1.1 kPa for 15 min.

After reaching their stationary growth phase, the cultures were harvested and the fermentation media was divided into two aliquots, one for organic extraction and the other for an aqueous fraction and SuperFluids fractionation of the cell pellet. The second aliquot was centrifuged at 8,000 g to collect the cell pellet, which was then lyophilized and transferred to an extraction cartridge. SuperFluids extraction was carried out with CO₂ and up to 30% methanol in an ISCO SFX 3560 automated extraction system equipped with an autosampler.

The cell pellet was first extracted with neat CO₂ to produce a S1 fraction. The sample was then depressurized and the disrupted cells re-extracted with neat CO₂ to produce a S2 fraction. The methanol concentrations in the CO₂ were then incrementally increased to 5, 10, 20, and 30% to produce S3, S4, S5 and S6 fractions. The supernatant from the centrifugation step was separated as an aqueous “fraction.” Organic fractions were prepared by adding 0.5 vol of butanol to the first aliquot in a 500 ml flask, and shaking it for 30 min at 250 rpm. The mixture was then centrifuged at 8,000 g for 10 mins, and the butanol phase was collected and stored at −20° C.

Organic solvent extraction was carried out on one half of the fermentation broth (250 ml) using conventional methods utilized in the pharmaceutical industry. Typically, 250 ml of the grown culture was extracted by adding 125 ml butanol to the 500 ml Erlenmeyer flask. The flasks were shaken at 250 rpm for 30 min, and were then allowed to stand for 30 minutes. Most of the lower aqueous layer was suctioned off with a 1 ml plastic pipette attached to a vacuum pump. The flask contents were transferred to centrifuge tubes which were centrifuged in a Sorvall RC2-B centrifuge at 8,000 g for 10 min to completely separate the phases. The upper butanol phase was collected by aspiration using a disposable Pasteur pipette, and transferred to a 15 ml polypropylene or glass storage tubes.

The other half or the fermentation broth (typically 250 ml) was used for an aqueous fraction, and SuperFluids CXF fractionation of the cell pellet. SuperFluids (SFS) are gases such as carbon dioxide, which when compressed, exhibit enhanced thermodynamic properties. Furthermore, the polarity of SFS can be adjusted by mixing in varying proportions of a cosolvent such as an alcohol. The second aliquot was centrifuged at 8,000×g to collect the cell pellet, which was then dried. SuperFluids CXF fractionations were carried out on an ISCO (Lincoln, Nebraska) SFX 3560 automated extractor. As shown in FIG. 1, this is a dual pump system, utilizing syringe pump 1 for neat critical fluid and syringe pump 2 for modifier.

The pumps are independently controllable, allowing easy adjustment of the fluid composition. The dried cell pellet was transferred to a 10 ml ISCO extraction cartridge, numbered 3 in FIG. 1, after which the cartridge was filled with 3 mm diameter glass beads to reduce the dead volume. After loading a cartridge on the cartridge holder, the fractionation procedure commenced. The system was brought to 3,000 psig and 40° C., and extracted for 10 minutes with pure CO₂. This fraction was collected in methanol in a glass vial, numbered 19 in FIG. 1. Next, rapid depressurization was carried out in order to disrupt the cells. Next, the fractionation parameters were set to: SFS CO₂ at 3,000 psig and extraction temperature 40° C., step extractions with methanol as cosolvent at 0, 5, 10, 20, and 50 vol %, each step being 10 min. Because some void volume remained between the glass beads, the composition of the extraction medium did not change sharply or immediately when modifier flowrate was adjusted to give a new fluid composition. Each sample thus yielded 6 fractions, which were collected in methanol in separate glass vials. The different collection vials are mounted in a carousel. The aqueous, butanol and SFS fractions were tested for antiplaque bioactivity.

### Example 4: Screening of Marine Microorganisms Fractions in Growth Inhibition Studies

The ability of the marine microorganism fractions and extracts to inhibit the growth of three orally relevant microorganisms, Haemophilus actinomycetemcomitans, Streptococcus mutans and Actinomyces viscosus (thought responsible for plaque formation, caries and juvenile peridontitis), were evaluated in 96-well microtiter plate format using a microtiter plate reader and visually confirmed. These assays identified twelve potential “hits” that exhibited greater that 95% inhibition of growth of the two Gram-positive bacteria, Streptococcus mutans and Actinomyces viscosus.

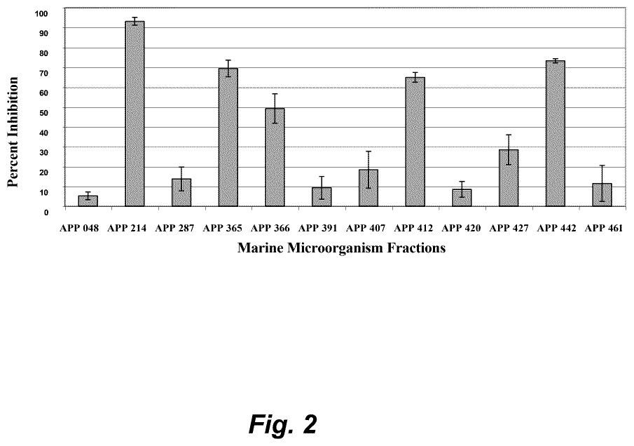

Subsequently, we tested these twelve potential “hits” for inhibition of growth against MDR Staphylococcus aureus ATCC 33592 (gentamicin and methicillin) and Enterococcus faecium ATCC 51559 (ampicillin, ciprofloxacin, gentamicin, rifampin, teicoplanin and vancomycin). Activity was determined based on inhibition of cell growth relative to a viability control (no added extract) on the same plate (FIG. 2). These were crude butanol extracts in which the absolute purity of the bioactive compound could range from 0.001 to 0.1%. The assay was based on “Methods for dilution antimicrobial susceptibility tests for bacteria that grow aerobically; approved standard-fifth edition M7-A5” (National Committee for Clinical Laboratory Standards, 2000).

Assess Biological Activity of Purified Active Component: Cultures of Actinomyces viscosus, Streptococcus mutans, and Haemophilus actinomycetemcomitans were obtained from the American Type Culture Collection (ATCC) following instructions provided by ATCC. Frozen stocks of each bacteria were prepared by adding 10% (v/v) volume of glycerol to an overnight culture; aliquots of the resulting suspension were then frozen at −80° C.

Minimum Inhibitory and Minimum Bactericidal Activity: Minimum inhibitory concentrations (MIC) were determined to identify the minimum concentration of a compound needed to inhibit the growth of the target organism. Minimum bactericidal concentrations (MBC) were determined to identify the minimum concentration of a compound needed to kill the target organism. The MBC was determined from the same plates set up for the MIC.

Cultures of Actinomyces viscosus ATCC #43146 and Haemophilus actinomycetemcomitans ATCC #43717 were initiated by inoculating 200 μl of each frozen stock culture into 5 ml Brain-Heart Infusion broth and incubating overnight at 37° C. in a humidity controlled 5% CO₂ incubator. Streptococcus mutans ATCC #25175 was inoculated (0.2 ml) from a frozen stock culture into 5 ml of Mueller Hinton II medium supplemented with 5% sucrose (MHS). A mixed population of “wild type” bacteria was initiated with tooth scrapings collected from a volunteer with a sterile applicator stick. The tooth scrapings were inoculated into MHS. All cultures were grown overnight at 37° C. with 5% CO₂. The concentration of each culture was adjusted to approximately 1×10⁷ colony forming units (CFU) per ml by diluting an aliquot of the overnight culture in the respective culture medium and reading the absorbance at 600 nm in a spectrophotometer. Each culture was diluted 1:100 in respective media and this was used as the stock suspension for the MIC assay. The concentration of each inoculum was verified by serial dilution in respective media and standard plate counts on Brain Heart Infusion Agar for A. viscosus, the wild type bacteria and S. mutans. H. actinomycetemcomitans was plated on Wilkins Chalgren agar. All plates were incubated at 37° C. with 5% CO₂ for 2-4 days

The APP214 purified compounds were prepared in DMSO at stock concentrations of 5 mg/ml. Crude fractions collected from the C18 column were tested at known volumes depending upon fraction size until the chromatography was scaled up enough to provide adequate biomass for a dry weight. Triclosan and chlorhexidine were prepared in DMSO at a stock concentration of 2.5 mg/ml. Seven dilutions (1:2) of each fraction were made in DMSO. This gave starting concentrations of 2.5 to 0.020 mg/ml for triclosan and chlorhexidine and 5.0 to 0.039 mg/ml for the APP214 purified compounds. Each dilution (2.5 μl) was tested in triplicate in a 96 well plate. 97.5 μl of the challenge organism suspension was added per well to give approximately 1×10⁵ CFU/well. Viability controls of 2.5 μl DMSO plus 97.5 μl of the cell suspension were prepared in 8 replicates. All plates were incubated at 37° C. with 5% CO₂ for 24±2 hours. All plates were read in a microtiter plate reader at 630 nm. All plates were also scored by eye to determine if the absorbance values are due to growth or precipitate formation. Each well was scored against the viability control.

The minimum bactericidal activity was determined from the MIC plates by selecting three concentrations in triplicate in which no bacterial growth was observed for either the APP214 purified compounds or the positive controls (Triclosan and Chlorhexidine). The contents of each the well were diluted 1:100 in respective medium and incubated at 37° C. with 5% CO₂ for 24-48 hours. All tubes were scored for growth both manually and by reading the absorbance at 630 nm after 24 hours.

Biofilm Assay: A biofilm assay was conducted to access the bactericidal activity of the target compounds when the challenge organisms are contained within a biofilm. Bacteria exist in biofilms on the tooth surface and they have increased resistance to antimicrobial agents in this state. Cultures of Actinomyces viscosus ATCC #43146 was initiated by inoculating 200 μl of each frozen stock culture into 5 ml Brain-Heart Infusion broth and incubating overnight at 37° C. in a humidity controlled 5% CO₂ incubator. Streptococcus mutans ATCC #25175 was inoculated (0.2 ml) from a frozen stock culture into 5 ml of Mueller Hinton II medium supplemented with 5% sucrose (MHS). All cultures were grown overnight at 37° C. with 5% CO₂. Biofilms were prepared by incubating dense 5 mm hydroxyapatite disks at 37° C. with 5% CO₂ for 2 hours with 50 μl of purified saliva. A two-hour incubation period allows maximum pellicle formation which is essential for the formation of the biofilm on the disc surface and mimics the formation of a natural biofilm in the mouth. Each disc was then individually inoculated with microorganisms at a starting concentration of 1×10⁶ colony forming units (CFU)/ml. The biofilms were grown for 24 hours. Disks with adherent biofilms were then transferred to wells of 24-well microtiter plate containing 2 ml PBS to remove unattached bacteria. After 5-10 min., biofilms were transferred to wells containing 300 μl PBS plus 200 μl of the active compound or control at desired concentration in DMSO in triplicate per organism. A viability control of DMSO was also prepared for each organism in triplicate. The 24-well plates were incubated for 1 hour at 37° C. in a humidity-controlled 5% CO₂ incubator. After incubation, the biofilms were transferred to a 24 well microtiter plate containing 2 ml PBS per well for 5-10 min., then to 50 ml conical tubes containing 10 ml PBS plus 0.1% Tween 80 (v/v) to aid in removal of the biofilm from the disc. The tubes were vortexed for 1-2 min to release adherent biofilms and generate a uniform suspension of organism. The suspensions were serially diluted (1:10) four to six times and plated on BHA. The plates were incubated at 37° C. in a humidity-controlled 5% CO₂ incubator for 2-4 days. The resulting bacterial colonies were counted to determine the number of organisms contained in each biofilm. The results were compared to the viability controls.

Six marine microorganisms that showed greater than 25% inhibition of one or both of these organisms (FIG. 2) were tested for minimum inhibitory concentration (MIC) and minimum bactericidal concentration (MBC). The most active extract, APP-214, had MICs of 15.6 μg/ml and 3.9 μg/ml and MBCs of 31.25 μg/ml and 15.63 μg/ml respectively against MDR Staphylococcus aureus and Enterococcus faecium. Assuming a molecular weight of 500 to 1,000 of the bioactive compound, the specific anti-MDR bioactivity is in the micromolar range. The activities reported here are for crude cell extracts, in which the absolute purities of the bioactive compounds may range from 0.001 to 0.1%. Then, the pure compounds have nanomolar bioactivities.

### Example 5: Identification of Bioactive Marine MicroOrganism and Bioactive Product

The marine microorganism fraction, APP 214, which produced the most bioactive anti-MDR compound was identified by fatty acid methyl ester analysis (FAME) on a Hewlett Packard Model 6890 Gas Chromatograph System with a MIDI database (Sasser et al., 1991). Based on primary similarity indices of 0.871 and 0.860, the marine microorganism was tentatively identified by FAME as Vibrio gazogenes.

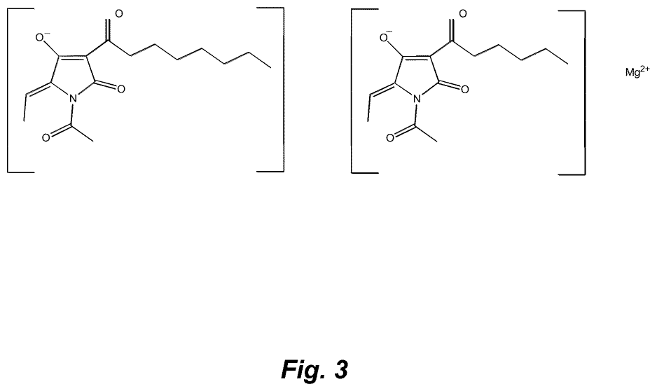

Subsequently, through bioassay fractionation using MIC of S. mutans and A. viscosus and in collaboration with Dr. D. John Faulkner, Scripps Institution of Oceanography, we isolated and purified the bioactive compounds (Magnesidin) of V. gazogenes by chromatographic techniques and identified the primary antimicrobial compound against to be Magnesidin (FIG. 3) by mass spectral and proton NMR analyses.

Magnesidin was isolated by bioassay-guided fractionation of APP-214 utilizing Gram-positive bacteria, Streptococcus mutans and Actinomyces viscosus. To date, Magnesidin has not been tested against MRSA and VRE. Magnesidin is suspected of being responsible for the anti-MDR activity of APP-214, a Gram-negative bacterium isolated offshore Plum Island, MA. There is a high likelihood that Magnesidin may be responsible for the anti-MDR bioactivity since reutericyclin, a structurally related tetramic acid, has been reported to have activity against MRSA and VRE. Reutericyclin was isolated from sourdough isolates of Lactobacillus reuteri, LTH2584 and is the first low molecular weight antibiotic from lactic acid bacteria.

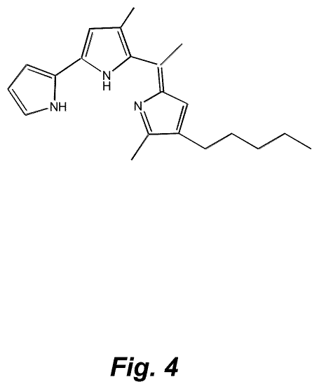

Vibrio gazogenes produces two anti-microbial compounds, Magnesidin and Prodigiosin (FIG. 4). Magnesidin is known to be effective against Gram-positive bacteria but its capacity against MDR strains has not been investigated. Magnesidin has an LD₅₀ in mice of 50 mg/kg (intraperitoneal) and 1,000 mg/kg (oral and subcutaneous).

The other compound, prodigiosin (FIG. 4) has exhibited antimicrobial, antifungal, cytotoxic and immunosuppressive activities but has not been developed because of high systemic toxicity.

### Example 6: Isolation and Purification of Magnesidin

Isolation of Magnesidin: Dried aqueous and butanol extracts of APP214R, APP214S, APP214T and APP214U (50 mg total) were mixed and run on a silica gel column and eluted with hexane:ethyl acetate:methanol gradient. Twenty-three fractions were collected from the silica gel column, and were monitored by TLC on silica gel F254 with hexane:acetone:methanol (2:1:0.1 v/v/v/) as the mobile phase. Similar fractions were combined and were run on a small C18 column with a mobile phase of methanol:H₂O (85:15 v/v). Thirty 20 ml fractions were collected and monitored by TLC on silica gel F254 with hexane:acetone:methanol (2:1:0.1 v/v/v/) as the mobile phase. Similar fractions were combined to provide a total of five fractions.

Fraction 1 contained four compounds (B1-B4) in addition to the red prodigiosin. Fraction 2 contained compounds B2, B3, and B4. Fractions 3-5 contain low levels of compounds other than the compound of interest. Fraction 1 was dried onto C18 and eluted with a mobile phase of methanol:Water (85:15 v/v). Thirty 20 ml fractions were collected. The two Magnesidin homologs were collected in semi-purified pure form from fractions 23-25. Compounds B3 and B4 were analyzed by NMR. Presumptive Magnesidin was isolated from fractions B3 and B4. These two fractions were sent to Dr John Faulkner for NMR analysis.

Fraction B3: The mass spectrum is almost identical to the published spectrum of the higher homolog (C6) of Magnesidin and confirms a positive identification. The ¹H and COSY NMR spectra conform the assignment but contains peaks that suggest the presence of an impurity that is either a Magnesidin containing a different hydrocarbon chain or a fatty acid.

Fraction B4: The ¹H NMR suggests that this is a lower homolog (C4) of Magnesidin with two less methylene groups in the side chain. This sample is >95% pure.

NMR Analysis: Fractions B3 (2.5 mg) and B4 (3.0 mg) were sent to John Faulkner at Scripps Oceanographic Institute for NMR analysis. Identification of chemical structures was accomplished by interpretation of spectral data, primarily ¹H NMR spectroscopy. Fractions were dried and dissolved in 800 μl of CDCl₃ or DMSO-d₆ and analyzed using a 400 MHz ¹H NMR spectrometer.

Isolation of Active Compound: The dried cell pellet was extracted in a Soxhlet apparatus for 15 hours with ethyl acetate. Ethyl acetate was used in place of butanol in this large-scale extraction. Magnesidin is preferentially soluble in ethyl acetate and this solvent is much easier to remove to allow concentration during purification. The extract was concentrated under vacuum and a dry weight was taken to calculate the yield. Although the initial isolation of this compound was done first on silica gel, this step was excluded from the second purification because it provided no separation of the red prodigiosin from the mixture. This red compound has bioactivity against the challenge organisms but much less than that of the semi-purified Magnesidin homologs. The extract was rotary evaporated onto a C18 polymer matrix (40 μm Baker bond) at a 5% load (weight per weight). The coated C18 was packed over a column of C18 polymer matrix (Bakerbond 40 μm). The column was eluted with a mobile phase of methanol:water (60:40 v/v). A mobile phase of 60% methanol was used because this eluted the Magnesidin homologs but did not move the red prodigiosin on the column. Fractions were monitored for the presence of the active compound by bioactivity and by HPLC.

Fractions were monitored for bioactivity by MIC against A. viscosus. Fractions were monitored by HPLC using a 25 cm C18 Cap cell column (5 μm Phenonomex) with a mobile phase of methanol:water:trifluoroacetic acid (50:50:0.05 v/v/v) and a photodiode array detector. Although these fractions were bioactive they contained negligible quantities of the two Magnesidin homologs therefore a second isolation procedure was used to target Magnesidin.

In order to optimize the isolation of the active compound, a second isolation was done on C18 under acidic conditions (pH 2.8, acetic acid). The extract was concentrated under vacuum and a dry weight was taken to calculate the yield. The extract was rotary evaporated onto a C18 polymer matrix (40 μm Baker bond) at a 5% load (weight per weight). The coated C18 was packed over a column of C18 polymer matrix (Bakerbond 40 μm). The column was washed with methanol:water (20:80 v/v, pH 2.8). The column was washed with 20% methanol in dH₂O to remove polar impurities. This concentration did not move the C4 or C6 homologs off the column but provided a level of separation from other compounds. The column was then eluted with 60% aqueous methanol (pH 2.8). Fractions were monitored for the presence of the active compound by bioactivity and by HPLC. Fractions were monitored for bioactivity by MIC against A. viscosus. Fractions were monitored by HPLC using a 25 cm C18 Cap cell column (5 μm Phenonomex) with a mobile phase of methanol:water:trifluoroacetic acid (50:50:0.05 v/v/v) and a photodiode array detector. Although these fractions contained milligrams quantities of both the C4 and C6 Magnesidin homologs they had no detectable bioactivity against A. viscous.

To narrow down the source of the bioactivity the C6 homolog was purified on a 25 cm C18 Cap cell column (5 μm Phenonomex) with a mobile phase of methanol:water:trifluoroacetic (50:50:0.05 v/v/v). The purified homolog was tested for bioactivity against Actinomyces viscosus, Haemophilus actinomycetemcomitans and a wild type culture.

### Example 8: Bioactivity of APP-214 Marine Microorganism Fraction from Vibrio gazogenes and Semi-Purified Compounds

Our results indicate that Vibrio gazogenes produces the two anti-microbial compounds Magnesidin (FIG. 3) and the red compound Prodigiosin (FIG. 4). Magnesidin was determined in the preliminary results to be more active than prodigiosin and was targeted as the lead compound. Magnesidin occurs as an equivalent mixture of the magnesium salts of 1-acetyl-3-n-hexanoyl-5-ethylidenetetramic acid (n=4) and 1-acetyl-3-n-octanoyl-5-ethylidenetetramic acid (n=6) [C₂₈H₃₆O₈N₂Mg+C₃₂H₄₄O₈N₂Mg]. Both homologs were isolated in pure form from APP-214.

Minimum Inhibitory Concentration of Magnesidin Homologs: The minimum inhibitory (MIC) and minimum bactericidal concentrations for each of the two purified homologs were tested against orally relevant microorganisms and a “wild type” culture. The MIC and MBC values for the purified compounds are given in Tables 2 and 3, respectively. Triclosan and Chlorhexidine were included in each assay as a positive control.

TABLE 2
Minimum Inhibition Concentration for C4 and C6 Magnesidin Homologs

| | MIC (μg/ml) | | | | |
|---|---|---|---|---|---|
| Organism | Triclosan | Chlorhexidine | | C4 Homolog | C6 Homolog |
| A. viscosus | 3.9 | 3.9 | | 15.63 | <0.75 |
| H. actinomycetemcomitans | <0.75 | 3.9 | | 62.5 | 62.5 |
| S. mutans | 7.8 | 1.95 | (0.98) | 125 | 3.9 |
| Wild Type | 7.8 | 7.8 | (7.81) | 31.25 | 7.8 |

TABLE 3
Minimum Bactericidal Concentration for C4 and C6 Magnesidin Homologs

| | MBC (μg/ml) | | | |
|---|---|---|---|---|
| Organism | Triclosan | Chlorhexidine | C4 Homolog | C6 Homolog |
| A. viscosus | 3.9 | 7.8 | 31.25 | <0.75 |
| H. actinomycetemcomitans | <0.75 | 3.9 | 125 | 125 |
| S. mutans | 15.6 | 1.95 | 62.5 | 7.8 |
| Wild Type | 15.6 | 15.6 | 125.0 | 15.6 |

The initial isolation gave only milligram amounts of Magnesidin. In order to continue the experiments more Magnesidin was isolated. In the second isolation procedure, the silica gel column was eliminated because this did not provide any separation of the two Magnesidin homologs from the red Prodigiosin. This step was important to narrow down the bioactivity. Fractions were collected from the C18 column and were assayed primarily for bioactivity. The results of the bioactivity data are presented in Table 4. These were later screened for the presence of the two Magnesidin homologs. The Magnesidin concentrations are given in Table 4. Although these fractions were active, they contained only trace concentrations of the Magnesidin homologs.

TABLE 4
Minimum Inhibitory Concentration of Selected C18 Column Fractions
and Magnesidin Concentrations Extracted without Acid

| Fraction | MIC (μg/ml) | C4 Homolog (μg/ml) | C6 Homolog (μg/ml) |
|---|---|---|---|
| G | 7.25 | 0.0 | 0.0 |
| H | 21.8 | 0.0 | 0.0 |
| K | 20.0 | 0.0 | 0.0 |
| L | 8.25 | 3.8 | 0.8 |
| M | 505 | 1.9 | 0.7 |
| N | 6.25 | 0.0 | 0.0 |

This suggested that Magnesidin was required in only trace concentrations or may not be the most bioactive compound. The bioactivity was spread over eight column void volumes. In order to concentrate the Magnesidin collected from the column into less fractions, the mobile phase was acidified to pH 2.8 with glacial acetic acid. Magnesidin is stable over a pH range of 2-8. The ethyl acetate extract was again dried onto C18 (40 μm Bakerbond) and washed first with 4 column void volumes of 20% aqueous methanol (pH 2.8) to remove polar impurities followed by 18 void volumes of 60% aqueous methanol (pH 2.8). For this experiment, the fractions were monitored for bioactivity against A. viscosus and Magnesidin concentration by HPLC. These fractions contained milligram concentrations of both Magnesidin homologs but were not bioactive. The results of the bioactivity are given in Table 5.

TABLE 5
Minimum Inhibitory Concentration of C18 Column Fractions
and Magnesidin Concentrations Extracted with Acid

| Fraction | MIC (μg/ml) | C4 Homolog (μg/ml) | C6 Homolog (μg/ml) |
|---|---|---|---|
| A | >2792.5 | 0.0 | 0.0 |
| B | >282.5 | 8.4 | 0.0 |
| C | >202.5 | 8.9 | 0.0 |
| D | >210.0 | 9.0 | 0.0 |
| E | >92.5 | 934.0 | 0.0 |
| F | >137.5 | 1371.0 | 0.0 |
| G | >42.5 | 386.9 | 5773.0 |
| H | >75.0 | 124.0 | 4160.0 |
| I | >50.0 | 34.9 | 478.0 |

The next step in the investigation of the source of the active compound was the purification of the C6 homolog from the semi-purified fraction. This was essential to rule out the presence of compounds that may inhibit the antimicrobial effect. The C6 homolog was chosen because it was initially reported as the more active of the two homologs and the chromatography suggested it would be easier for the two homologs to separate. Fraction G listed in Table 5 was used as source material for the purification. The C6 homolog was isolated by repeated injection and fractionation on the HPLC. The combined fractions were dried and weighed. One (1) mg of the purified C6 homolog was collected. This was 98.9% chromatographically pure. The purified C6 homolog was tested for bioactivity against A. viscosus. The purified C6 homolog was not bioactive (see Table 6) at the highest concentration tested (125 g/ml). The semi-purified Magnesidin reported in Table 1 had an activity of <0.75 μg/ml. This number would be expected to become lower as the fraction was purified.

TABLE 6
Minimum Inhibition Concentration for
Purified C6 Magnesidin Homolog.

MIC (μg/ml)

| Organism | Triclosan | C6 Homolog |
|---|---|---|
| A. viscosus | 3.91 | >125 |
| H. actinomycetemcomitans | <0.49 | >125 |
| Wild Type | 7.81 | >125 |

This suggests that the C6 homolog is not the active compound and the active compound is another component in the semi-purified mixtures.

Microbial Biofilm Assay: The effectiveness of the two original semi-purified Magnesidin homologs were determined in a biofilm assay. This assay is expected to provide more accurate information on the activity of the compounds because bacteria occur in the mouth as biofilms. These biofilms consist of a consortia of organisms which have higher resistance to the antimicrobial agent than the individual organisms grown axenically.

TABLE 7
Log Reduction Values for Semi-purified C6 Magnesidin
Homolog in A. viscosus Biofilm Experiment

| A. viscosus | CFU/ml | | | | | Log10 | Log |
|---|---|---|---|---|---|---|---|
| Compound | 1 | 2 | 3 | Average | Std. Dev. | CFU/ml | Reduction |
| Viability | 9.2E+07 | 8.0E+07 | 7.4E+07 | 8.2E+07 | 9.2E+06 | 7.91 | N/A |
| Triclosan | 5.7E+07 | 4.5E+07 | 5.8E+07 | 5.3E+07 | 7.2E+06 | 7.73 | 0.19 |
| Chlorhexidine | 6.6E+07 | 6.6E+07 | 6.4E+07 | 6.5E+07 | 1.2E+06 | 7.82 | 0.10 |
| APP 214 | 6.2E+07 | 6.1E+07 | 8.0E+07 | 6.8E+07 | 1.1E+07 | 7.83 | 0.08 |
| Extract | | | | | | | |

TABLE 8
Log Reduction Values for Semi-purified C6 Magnesidin
Homolog in S. mutans Biofilm Experiment

| S. mutans | CFU/ml | | | | | Log10 | Log |
|---|---|---|---|---|---|---|---|
| Compound | 1 | 2 | 3 | Average | Std. Dev. | CFU/ml | Reduction |
| Viability | 1.1E+06 | 9.1E+05 | 1.3E+05 | 7.1E+05 | 5.1E+05 | 5.85 | N/A |
| Triclosan | 4.3E+05 | 8.3E+05 | 1.5E+05 | 4.7E+05 | 3.4E+05 | 5.67 | 0.18 |
| Chlorhexidine | 1.5E+05 | 5.0E+03 | 0.0E+01 | 5.2E+04 | 8.5E+04 | 4.71 | 1.14 |
| APP 214 | 6.9E+05 | 1.1E+06 | 6.8E+05 | 8.2E+05 | 2.4E+05 | 5.92 | −0.06 |
| Extract | | | | | | | |

Vibrio gazogenes is known to produce two antimicrobial compounds. Prodigiosin is a red compound which has both antimicrobial and antifungal activity. This compound has not been developed due to toxicity problems. The second antimicrobial compound Magnesidin is an equivalent mixture of the magnesium salts of 1-acetyl-3-n-hexanoyl-5-ethylidenetetramic acid (n=4) and 1-acetyl-3-n-octanoyl-5-ethylidenetetramic acid (n=6) [C₂₈H₃₆O₈N₂Mg+C₃₂H₄₄O₈N₂Mg]. Although this compound was initially isolated in our work and thought to be the active compound further purification of the C6 Magnesidin homolog gave no activity.

### Example 8: Identification of Other Bioactive Marine MicroOrganism and Bioactive Product

Five (5) other marine microorganisms, APP-365, APP-366, APP-412, APP-427 and APP-442, that had >25% inhibition of MRSA and VRE were also selected for further investigation in this research. These were tested as crude extracts (2.5 μg/ml) and may have a wide variation in the concentration of the bioactive compound so that APP-427 with an MRSA inhibition of ˜30% may be more bioactive than the crude extract of APP-214 with an MRSA inhibition of ˜30% after isolation and purification of the bioactive compounds.

Thus, six (6) marine microorganisms extracts have demonstrated moderate to significant inhibition of MDR Staphylococcus aureus ATCC 33592 (gentamicin and methicillin) and Enterococcus faecium ATCC 51559 (ampicillin, ciprofloxacin, gentamicin, rifampin, teicoplanin and vancomycin).

It is intended that the matter contained in the preceding description be interpreted in an illustrative rather than a limiting sense.

What is claimed is:

1. A marine microorganism fraction APP 214 extracted utilizing supercritical fluids from a saline fermentation broth of a marine microorganism effective against multi-drug resistant bacteria.

2. The multi-drug resistant (MDR) bacteria of claim 1 consisting of Staphylococcus aureus and Enterococcus faecium.

3. The marine microorganism fraction of claim 1 having a minimum inhibitory concentration (MIC) of 5-30 μg/mL against MDR Staphylococcus aureus.

4. The marine microorganism fraction of claim 3 having a minimum inhibitory concentration (MIC) of 15.6 μg/mL against MDR Staphylococcus aureus.

5. The marine microorganism fraction of claim 1 having a minimum bactericidal concentration (MBC) of 10-50 μg/mL against MDR Staphylococcus aureus.

6. The marine microorganism fraction of claim 5 having a minimum bactericidal concentration (MBC) of 31.25 μg/mL MDR against Staphylococcus aureus.

7. The marine microorganism fraction of claim 1 having a minimum inhibitory concentration (MIC) of 5-10 μg/mL against MDR Enterococcus faecium.

8. The marine microorganism fraction of claim 7 having a minimum inhibitory concentration (MIC) of 3.9 μg/mL against MDR Enterococcus faecium.

9. The marine microorganism fraction of claim 1 having a minimum bactericidal concentration (MBC) of 5-50 μg/mL against MDR Enterococcus faecium.

10. The marine microorganism fraction of claim 9 having a minimum bactericidal concentration (MBC) of 15.63 μg/mL against MDR Enterococcus faecium.

11. The marine microorganism of claim 1 that is Vibrio gazogenes.

12. The supercritical fluids of claim 1 that includes carbon dioxide.

13. The supercritical fluids of claim 1 that includes a mixture of carbon dioxide and a polar cosolvent.

14. The polar cosolvent of claim 13 that includes methanol and ethanol.

15. The supercritical fluids of claim 1 that operate at a pressure between 1,000 psig and 6,000 psig.

16. The supercritical fluids of claim 15 that operate at a pressure of 3,000 psig.

17. The supercritical fluids of claim 1 that operate at a temperature between 10° C. and 60° C.

18. The supercritical fluids of claim 17 that operate at a temperature of 40° C.

19. A mixture of compounds extracted from the marine microorganism fraction Vibrio gazogenes utilizing supercritical fluids from a saline fermentation broth of the marine microorganism effective against multi-drug resistant bacteria.

20. The mixture of claim 18 that does not include Magnesidin and Prodigiosin.
